(19) United States
(12) Patent Application Publication
(10) Pub. No.: US 2006/0100603 A1
(43) Pub. Date: May 11, 2006

(54) Medicine dispensing device for bottle fed patients

(76) Inventor: Stephen Mark Thwaits, Vernon Hills, IL (US)

Correspondence Address: Stephen Mark Thwaits, 2380 Alston Drive, Atlanta, GA, 30317, US

(21) Appl. No.: 10/896,132

(22) Filed: Jul. 22, 2004

(51) Int. Cl. A61M 5/00; A61M 25/00; A61M 39/10

(52) U.S. Cl. 604/533; 604/264; 604/187

(57) ABSTRACT

A device for administering medicine from a syringe into the lumen of a bottle nipple for dilution with a liquid from a bottle and ingestion by a bottle fed patient. The device being a length of dispensing tube attached to a conical syringe connector or luer syringe connector which can be attached to any sized cylindrical or luer syringe. The dispensing tube being inserted through a fissure at a base of a bottle nipple into the lumen of the nipple. Medicine being delivered from the syringe through the lumen of the syringe connector through the lumen of the dispensing tube to the lumen of the bottle nipple. The medicine being diluted at a controlled rate with a liquid from the bottle attached to the nipple and orally ingested by the patient through the normal sucking action.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION

The invention relates generally to the field of administering doses of liquid medication from a syringe to the lumen of a bottle nipple. The device allows the rate of delivery of the medication and the dilution of the medication to be independently controlled. The device allows the medicine to be disguised by being diluted and ingested with a tasty liquid.

The dispensing of medicine to adults, children, toddlers, infants and bottle fed patients is a common challenge with which many parents and practitioners are faced. Frequently these medicines are not palatable when dispensed in an undiluted medium. Once the undiluted medicine has been tasted, the second dose of medicine is rejected. This results in medicine not being ingested in the dose required. In an attempt to ensure that the full dose is ingested, additional medicine is dispensed to compensate for any spilled medicine. This could result in either a serious over-dose or under-ingestion of medicine.

Patients, especially young human patients such as infants, toddlers and children are comfortable with the particular shape and feel of the bottle nipple that they use for regular feeding. They may reject the introduction of a foreign nipple. Diluting the medicine in any tasty liquid, such as cow, soy or mother's milk, infant or child formula, juice, or any pleasant tasting liquid significantly increases the chances of ingestion.

Devices and methods have been developed to promote the repeated ingestion of diluted medicine. The ability to control the rate of medicine administration and dilution of the medicine is important in ensuring the effective delivery of medicine. Patients, especially young human patients such as infants, toddlers and children are comfortable with the particular shape and feel of the bottle nipple that they use for regular feeding. An unfamiliar nipple increases the risk that the nipple is rejected and the medicine is not ingested.

U.S. Pat. No. 5,487,750 (Burchett, et. al.) describes an integrated feeding bottle and liquid medication dispensing apparatus using a bottle nipple specific to the apparatus. It utilizes a deformable plastic bag with a sleeve for receiving a syringe. The Burchett design presents an unfamiliar nipple significantly increasing the risk of nipple rejection. The size and unfamiliar sight of the device aggravates an already disruptive process. The design of the device requires complicated manufacturing processes.

Still another reference, U.S. Pat. No. 4,821,895 (Roskilly), describes a feeding bottle accessory comprising a teat holder and bottle closure including a mounting. The attachment comprising a threaded cap that sets the nipple off-center from the axis of the bottle. The Roskilly device replaces the cap and nipple that the infant typically uses. This presents the patient with a large and unfamiliar nipple and does not allow for the controlled dilution of medicine.

Still another reference, U.S. Pat. No. 3,682,344 (Lopez), discloses a small, flexible enclosure on the exterior of the nipple itself. The Lopez design presents an unfamiliar nipple and does not provide any dilution of the liquid or allow the rate and volume of medicine dispensing to be controlled.

There are a variety of other pacifier applicators that dispense undiluted medicine. See U.S. Pat. No. 5,176,705 (Noble); U.S. Pat. No. 5,078,734 (Noble); U.S. Pat. No. 3,426,755 (Clegg). U.S. Pat. No. 5,129,532 (Martin) describes a nipple with a bottom wall housing containing an inlet in which medicine can be dispensed. Undiluted medicine is introduced which significantly increase the likelihood of rejection due to the bad taste of the medicine. The rate that the medicine is dispensed cannot be controlled. The design also introduces an unfamiliar pacifier nipple to the infant and increases the chance of rejection due to familiarity. Accordingly, several objects and advantages of my invention are:
a) to allow the dispensing of medicine through any bottle nipple, specifically the nipple that the infant, toddler, child and patient is accustomed to using. The familiar nipple increases the likelihood of the successful ingestion of the medicine;
b) the syringe connector allows the device to be connected to a syringe; thereby allowing the amount of medicine being dispensed to be monitored and controlled throughout the process;
c) the conical syringe connector allows the device to be connected to conical syringes of varying sizes; thereby increasing the range of syringes that can be used and enabling a wide range of medicine volume that can be dispensed per occasion;
d) the luer syringe connector allows the device to be connected to luer syringes of varying sizes; thereby increasing the range of syringes that can be used and enabling a wide range of medicine volume that can be dispensed per occasion;
e) to deliver the medicine to the lumen of the nipple allowing the medicine to be diluted with a tasty liquid prior to ingestion of the medicine, thereby reducing the risk of the medicine being rejected;
f) to enable the degree of dilution of the medicine with a tasty liquid to be controlled by the individual dispensing the medicine, thereby reducing the risk of the medicine being rejected;
g) to enable the rate of ingestion to be controlled by the individual dispensing the medicine, thereby reducing the risk of the medicine being rejected;
h) the small size of the device reduces the likelihood that the introduction of a foreign device into the normal feeding routine will be noticed; thereby reducing the chances of the bottle, nipple or medicine being rejected;
i) the device is reusable;
j) the simple design of the device allows it to be easily sterilized;
k) the device is simple and inexpensive to manufacture.

### BRIEF DESCRIPTION OF THE SEVERAL VIEWS OF THE DRAWING

FIG. 1A—shows a dispensing tube

FIG. 1B—shows a conical syringe connector

FIG. 1B—shows the dispensing tube and conical syringe connector constructed as one piece

FIG. 1D—demonstrates the usage of the device; attached to a conical syringe and inserted into the lumen of a generic nipple

FIG. 2A—shows a dispensing tube

FIG. 2B—shows a luer syringe connector

FIG. 2C—shows the dispensing tube and luer syringe connector constructed as one piece

FIG. 3A—shows a dispensing tube with an enlarged inlet end

FIG. 3B—shows a conical syringe connector

FIG. 3C—shows a dispensing tube threaded through a conical syringe connector and assembled as one piece

### DRAWINGS—REFERENCE NUMERALS

10—dispensing tube
15—outlet end of the dispensing tube
20—conical syringe connector
25—outlet end of the conical syringe connector
27—discharge end of generic cylindrical syringe
30—inlet end of conical syringe connector
35—cylindrical syringe
45—bottle nipple
50—fissure in bottle nipple base
55—lumen of bottle nipple
60—luer syringe connector
65—outlet end of luer syringe connector
70—inlet end of dispensing tube

### DETAILED DESCRIPTION OF THE INVENTION

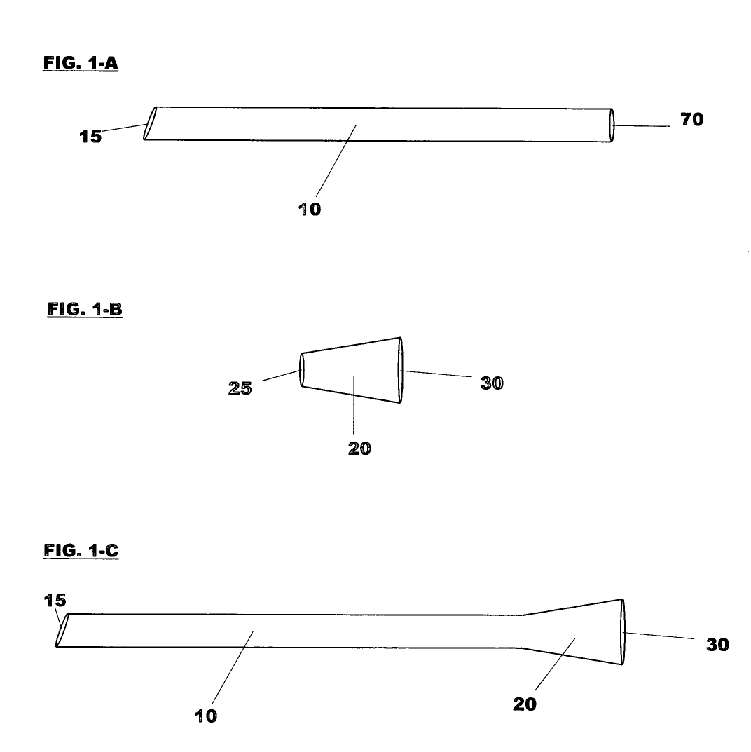

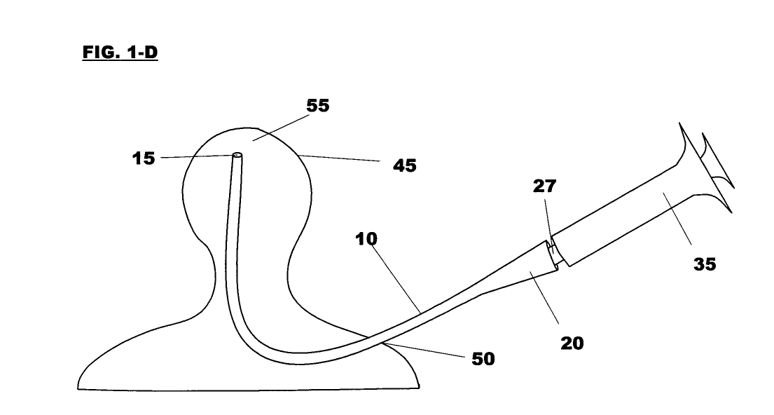

FIGS. 1A-FIG. 1D show a preferred embodiment. FIG. 1A shows a dispensing tube 10. The dispensing tube 10 having a lumen. The dispensing tube 10 having an inlet end 70 and an outlet end 15. The outlet end 15 having an angle. The dispensing tube 10 can be flexible, yet unyielding to prevent collapse of the dispensing tube 10 during medicine dispensing. The angle of the outlet end 15 facilitates easy insertion of the dispensing tube 10 through a fissure in a wall of a bottle nipple. Materials such as Polypropylene, Polypropylene copolymer, non-DEHP PVC, Teflon, Silicon and other materials with similar properties can be used to manufacture the dispensing tube 10. The dispensing tube 10 can be manufactured using a variety of processes such as extrusion and molding.

FIG. 1B shows a conical syringe connector 20 having a generally conical shape. The conical syringe connector 20 having a lumen. The conical syringe connector 20 having an inlet end 30 and an outlet end 25. The inlet end 30 having an internal diameter and the outlet end 25 having an internal diameter. The internal diameter of the inlet end 30 being greater than the internal diameter of the outlet end 25. The conical syringe connector 20 can be flexible. Materials such as Polypropylene, Polypropylene copolymer, non-DEHP PVC, Teflon, Silicon and other materials with similar properties can be used to manufacture the conical syringe connector 20. The conical syringe connector 20 can be manufactured using a variety of processes such as flow molding, injection molding and blow molding.

FIG. 1C shows the dispensing tube 10 and the conical syringe connector 20 constructed as one piece such that the lumen of the inlet end 70 of the dispensing tube 10 being in fluid contact with the lumen of the outlet end 25 of the conical syringe connector 20.

FIG. 1D demonstrates how the invention works. A discharge end 27 of a cylindrical syringe 35 being inserted into the lumen of the inlet end 30 of the conical syringe connector 20. The flexible nature of the conical syringe connector 20 enables it to expand and seal to the discharge end 27 of the cylindrical syringe 35. The outlet end 15 of the dispensing tube 10 being inserted through an existing fissure 50 in a base of a bottle nipple 45 such that the outlet end 15 being presented into a bottle nipple lumen 55 of the bottle nipple 45. A medicine being dispensed from the cylindrical syringe 35, through the lumen of the conical syringe connector 20 and the lumen of the dispensing tube 10 into the lumen 55 of the bottle nipple for dilution with a liquid being consumed.

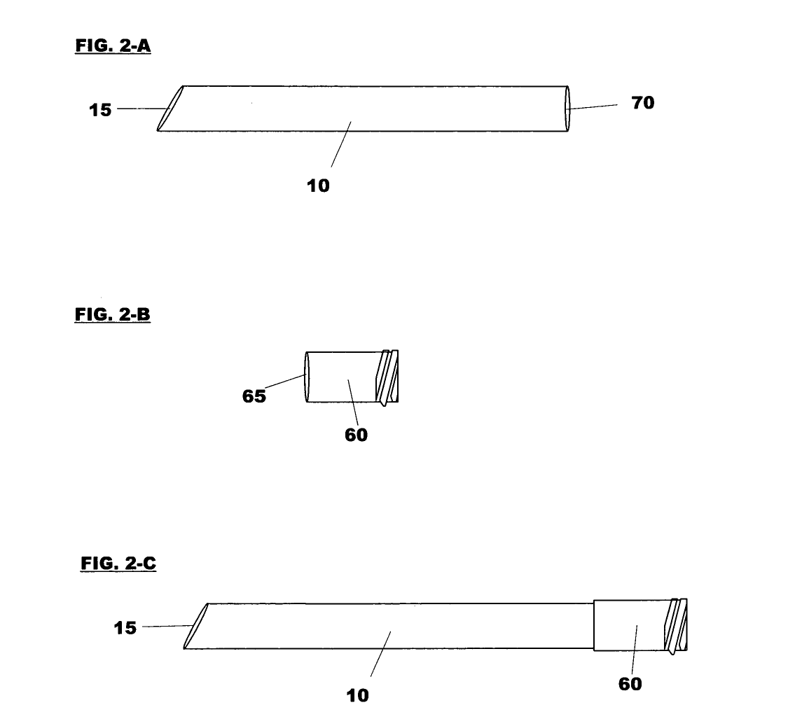

FIGS. 2A-FIG. 2C represent an additional embodiment of the invention. FIG. 2A shows a dispensing tube 10. The dispensing tube 10 having a lumen. The dispensing tube 10 having an inlet end 70 and an outlet end 15. The outlet end 15 having an angle. The dispensing tube 10 can be flexible, yet unyielding to prevent collapse of the dispensing tube 10 during medicine dispensing. The angle of the outlet end 15 facilitates easy insertion of the dispensing tube 10 through a fissure in a wall of a bottle nipple. Materials such as Polypropylene, Polypropylene copolymer, non-DEHP PVC, Teflon, Silicon and other materials with similar properties can be used to manufacture the dispensing tube 10. The dispensing tube 10 can be manufactured using a variety of processes such as extrusion and molding.

FIG. 2B shows a luer syringe connector 60 having a lumen and an outlet end 65. Those skilled in the art can readily select a luer connector to be used and can modify the connector to suit a particular luer syringe.

FIG. 2C shows the dispensing tube 10 and the luer syringe connector 60 constructed as one piece such that the lumen of the inlet end 70 of the dispensing tube 10 being in fluid contact with the lumen of the outlet end 65 of the luer syringe connector 60. The luer syringe connector 60 enables the dispensing tube 10 to be connected to a luer syringe.

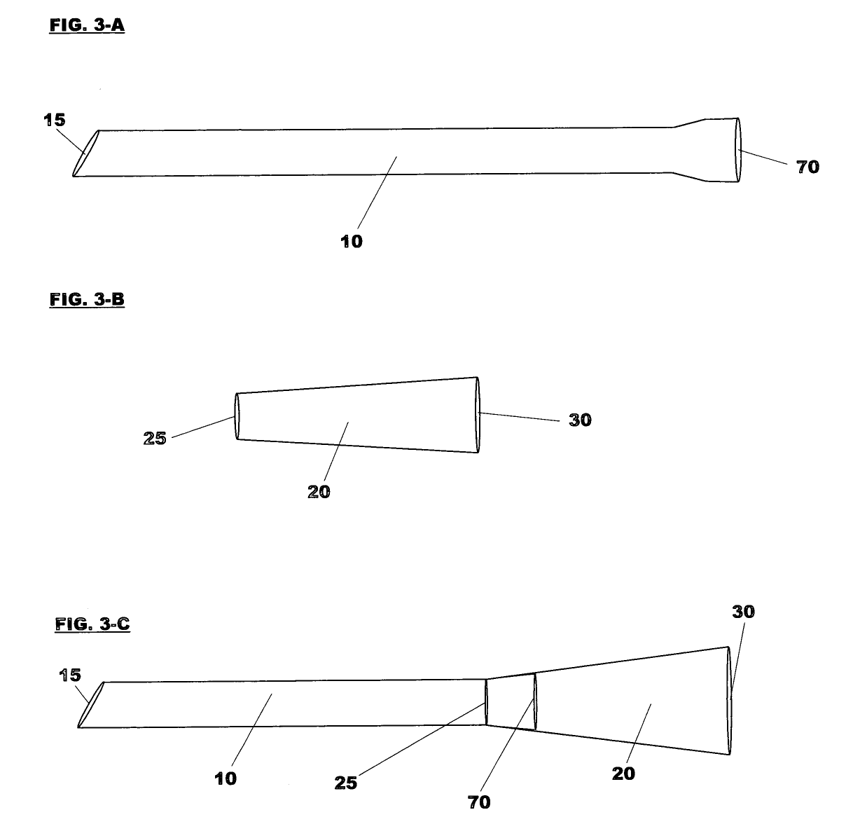

FIGS. 3A-FIG. 3C represent an additional embodiment of the invention. FIG. 2A shows a dispensing tube 10. The dispensing tube 10 having a lumen. The dispensing tube 10 having an external diameter, an inlet end 70 and an outlet end 15. The inlet end 70 having an external diameter. The outlet end 15 having an angle. The dispensing tube 10 can be flexible, yet unyielding to prevent collapse of the dispensing tube 10 during medicine dispensing. The angle of the outlet end 15 facilitates easy insertion of the dispensing tube 10 through a fissure in a wall of a bottle nipple. Materials such as Polypropylene, Polypropylene copolymer, non-DEHP PVC, Teflon, Silicon and other materials with similar properties can be used to manufacture the dispensing tube 10. The dispensing tube 10 can be manufactured using a variety of processes such as extrusion and molding.

FIG. 3B shows a conical syringe connector 20 having a generally conical shape. The conical syringe connector 20 having a lumen. The conical syringe connector 20 having an inlet end 30 and an outlet end 25. The inlet end 30 having an internal diameter and the outlet end 25 having an internal diameter. The internal diameter of the inlet end 30 being greater than the internal diameter of the outlet end 25. The conical syringe connector 20 can be flexible. Materials such as Polypropylene, Polypropylene copolymer, non-DEHP PVC, Teflon, Silicon and other materials with similar properties can be used to manufacture the conical syringe connector 20. The conical syringe connector 20 can be manufactured using a variety of processes such as flow molding, injection molding and blow molding.

FIG. 3C demonstrates a result of threading the dispensing tube 10 through the inlet end 30 of the conical syringe connector 20 and the outlet end 25. The external diameter of the dispensing tube 10 being equal to the internal diameter of the outlet end 25 of the conical syringe connector 20. The external diameter of the inlet end 70 of the dispending tube 10 being greater than the internal diameter of the outlet end 25 of the conical syringe connector 20. The external diameter of the inlet end 70 being greater than the internal diameter of the outlet end 25 prevents the dispensing tube 10 from being threaded through the outlet end 25. When assembled the device represents an alternative embodiment shown in FIG. 3C.

What I claim as my invention is:

1. A device comprising:
a dispensing tube
the said dispensing tube having a lumen
the said dispensing tube having an inlet end
the said dispensing tube having an outlet end
the said dispensing tube outlet end having an angle
a conical syringe connector
the said conical syringe connector having a lumen
the said conical syringe connector having an inlet end
the said conical syringe connector inlet end having an internal diameter
the said conical syringe connector having an outlet end
the said conical syringe connector outlet end having an internal diameter
the said dispensing tube and the said conical syringe connector constructed as one piece such that the lumen of the said inlet end of the said dispensing tube being in fluid contact with the lumen of the said outlet end of the said conical syringe connector.

2. A device comprising:
a dispensing tube
the said dispensing tube having a lumen
the said dispensing tube having an inlet end
the said dispensing tube having an outlet end
the said dispensing tube outlet end having an angle
a luer syringe connector
the said luer syringe connector having a lumen
the said luer syringe connector having an outlet end
the said dispensing tube and the said luer syringe connector constructed as one piece such that the lumen of the said inlet end of the said dispensing tube being in fluid contact with the lumen of the said outlet end of the said luer syringe connector.

3. A device and method for assembly comprising:
a dispensing tube
the said dispensing tube having a lumen
the said dispensing tube having an external diameter
the said dispensing tube having an inlet end
the said dispensing tube inlet end having an external diameter
the said dispensing tube having an outlet end
the said dispensing tube outlet end having an angle
a conical syringe connector
the said conical syringe connector having a lumen
the said conical syringe connector having an inlet end
the said conical syringe connector inlet end having an internal diameter
the said conical syringe connector having an outlet end
the said conical syringe connector outlet end having an internal diameter
the said dispensing tube and the said conical syringe connector constructed as separate pieces a method for assembling the device as in claim 3 comprising the steps as follows:
the said outlet end of the said dispensing tube being threaded through the said inlet end of the said conical syringe connector
the said outlet end of the said dispensing tube being pulled through the said outlet end of the said conical syringe connector
the external diameter of the said dispensing tube being equal to the internal diameter of the said conical syringe connector
the external diameter of the said inlet end of the said dispensing tube being greater than the internal diameter of the said outlet end of the said conical syringe connector means for sealing the lumen of the said dispensing tube with the lumen of the said conical syringe connector.
